(19) United States
(12) Patent Application Publication
(10) Pub. No.: US 2016/0351936 A1
(43) Pub. Date: Dec. 1, 2016

(54) SUBSTRATE-POWERED ENERGY STORAGE AND GENERATION SYSTEM

(71) Applicants: Keegan Cooke, Menlo Park, CA (US); Kevin Rand, New York, NY (US)

(72) Inventors: Keegan Cooke, Menlo Park, CA (US); Kevin Rand, New York, NY (US)

(21) Appl. No.: 15/072,340

(22) Filed: Mar. 16, 2016

Related U.S. Application Data
(60) Provisional application No. 62/134,020, filed on Mar. 17, 2015.

(51) Int. Cl. H01M 8/16; C12N 1/20; H01M 8/04992

(52) U.S. Cl. CPC H01M 8/16; H01M 8/04992; C12N 1/20

(57) ABSTRACT

The invention is a substrate-powered energy storage and generation system employing microbial fuel cell technology utilizing electrodes located or placed within and/or on a substrate comprising soil and/or other material combined with at least one liquid in a specialized assembly to create an oxygen rich and oxygen poor environment surrounding the electrodes to create a voltage potential capable of producing sufficient current to power at least one electrical load.

### CROSS-REFERENCE TO RELATED APPLICATIONS

This application claims priority to U.S. Provisional Application No. 62/134,020, filed Mar. 17, 2015, which is incorporated herein by reference for all purposes.

### BACKGROUND OF THE INVENTION

1. Field of the Invention

The invention is in the field of power generation, more specifically the field of power generation using electrodes located within a substrate, enabling ion transport and corresponding current flow through an external load.

2. Description of the Prior Art

Microbial fuel cells (MFCs) are bio-electrical devices that harness the natural metabolisms of microbes to produce electrical power directly. Within the MFC, microbes act as a catalyst to break down sugars and other nutrients in their surrounding environment and release a portion of the energy contained within those molecules in the form of electricity. MFCs remained a laboratory curiosity for nearly a century before the major advances in biochemistry, microbiology, and low-power devices in the last decade stimulated significant interest in their commercial potential. This interest has led to the development of environmental MFCs which harness the energy contained within substrates of the natural world, such as soils or sediments or compost or food waste. Over the last several years, the scientific understanding of various MFC processes has been greatly enhanced and power generation capability of MFCs, both in the lab and in the field, has risen steadily. The first deployment of an environmental MFC was demonstrated in 2007 by Lenny Tender of the Naval Research Laboratory (NRL), in which an ocean sediment-based MFC was used to power a meteorological buoy. Soil-based MFCs were first explored in 2006, and it was demonstrated that power could be generated from the microbes and the nutrients found within the soil alone. Thus substrate-powered energy storage and generation exploration has begun and has been demonstrated. The present invention is an important advancement in the field.

### SUMMARY OF THE INVENTION

In at least one embodiment, the invention is a substrate-powered energy storage and generation system employing microbial fuel cell technology utilizing a specialized assembly to power at least one electrical load, comprising at least one sealable or non-sealable vessel, a means of enclosing at least one sealable vessel, at least one fuel and/or substrate and/or electrolyte within the sealable or non-sealable vessel, at least one anode in at least one first location within the fuel and/or substrate and/or electrolyte within the sealable or non-sealable vessel, at least one means to attach at least one anode wire to at least one anode, at least one cathode in at least one second location within the sealable or non-sealable vessel, at least one means to attach at least one cathode wire to at least one cathode, a means of passing at least one anode wire and at least one cathode wire through the means of enclosing at least one sealable or non-sealable vessel, a means of attaching at least one cathode wire to at least one plus terminal and a means of attaching at least one anode wire to at least one minus terminal.

The invention can further comprise at least one electrical load placed between at least one plus terminal and at least one minus terminal.

At least one anode and at least one cathode can exhibit at least one different redox potential.

At least one anode can be placed in a nutrient-rich oxygen-poor environment and at least one cathode can be placed within an oxygen-rich environment.

The oxygen-poor environment can either include at least one of Shewanella bacteria, Geobacter bacteria, Deltaproteobacteria, Clostridia, and/or at least one other bacteria and/or is created, facilitated, and/or enriched by the addition of at least one of Shewanella bacteria, Geobacter bacteria, Deltaproteobacteria, Clostridia, and/or at least one other bacteria.

The oxygen-poor environment can be located at least one depth under soil and/or at least one fuel and/or substrate and/or electrolyte and the oxygen-rich environment can be positioned in at least one location exposed to the air and or on top of soil and/or at least one substrate.

The electrical load can be at least one of at least one LED, desk lamp, night light, mood lighting, clock, thermometer, moisture sensor, temperature sensor, pressure sensor, sound sensor, other sensor or combination of sensors, buzzer, audio circuit, radio transmitter and/or receiver, Bluetooth transmitter and/or receiver, a capacitor, a resistor, an active circuit, a rechargeable battery, voltage step-up circuit or “Joule-thief” circuit acting as an intermediary sub-system interposed between the substrate-powered energy storage and generation system and at least one electrical load.

At least one fuel and/or substrate and/or electrolyte can be at least one of soil, mud, soil and at least one liquid, garbage and/or bio-degradable garbage, compostable material, compost starter, microbe and/or bacteria-rich soil, soil with vegetation growing within, chemicals, rotting organic and/or inorganic material, human and/or animal waste, guano, minerals, oil and/or oil byproducts, chemical spills on seawater, lake water, and/or water of any mixture, and/or toxic waste.

At least one anode wire and at least one cathode wire can be at least one of titanium, a titanium alloy, a non-reactive conductor, conductive ink, gold, copper, tin, aluminum, steel, stainless steel, and/or other conductive alloy, mixture, suspension, material, or substance.

At least one anode and at least one cathode can be made from at least one of graphite, carbon felt, graphite felt, carbon cloth, graphite cloth, carbon paper, graphite paper, reticulated carbon foam, reticulated graphite foam, aluminum, steel, and/or stainless steel.

At least one means to attach at least one anode wire to said at least one anode is weaving, threading, and/or puncturing at least one wire through said at least one anode in at least one place and said at least one means to attach at least one cathode wire to said at least one cathode is weaving, threading, and/or puncturing at least one wire through said at least one cathode in at least one place.

Increasing the contacting surface area between the wire and the electrode reduces the overall resistance experienced by electrons flowing between the wire and the electrode, which is important for the efficiency of the system. Thus, weaving, threading, and/or puncturing the electrode with the bare wire lowers the resistance of this union of two materials, and the voltage drop and corresponding loss in efficiency. Resistance multiplied by current equals voltage, and this voltage is the voltage drop that lowers deliverable power and efficiency of any power transmission system.

The invention can further comprise at least one voltage boosting and/or voltage step-up circuit to produce at least one voltage that is higher than the voltage produced between said at least one anode and said at least one cathode. This includes the use of a charge-pump integrated circuit, such as the Seiko S-882Z series for an example, as and/or the use of a “Joule-thief” circuit comprising of capacitors, transistors, ferrite toroids, coiled wire, inductors, and/or LEDs or other loads.

The invention can further comprise a low-power circuit that either produces at least one wireless transmission of information comprising at least one parameter and/or produces a blinking LED representative of at least one attribute, and at least one attribute can be a function of at least one parameter, and at least one attribute can be at least one of LED on time, LED off time, LED brightness during the LED on time, LED frequency and period, wherein the frequency is the mathematical reciprocal of the period, and the period is the mathematical sum of the LED on time and the LED off time, and wherein at least one parameter can be at least one of open cell voltage under no load, voltage under load, voltage increase and/or voltage decrease, current delivered to at least one load, and/or power delivered to at least one load.

The invention can further comprise an application comprising software to run on at least one smart device, using at least one capability of the smart device, with the application working in conjunction with and supplemental to the substrate-powered energy storage and generation system to provide at least one status indication and/or indication of at least one parameter and/or at least one attribute and/or to store information and to make accessible and display at least one of the following: at least one parameter, at least one parameter as a function of time, the mathematical integral of at least one parameter as a function of time between at least one first time and at least one second time, other information associated with the substrate-powered energy storage and generation system.

The means of passing at least one anode wire and at least one cathode wire through the means of enclosing at least one sealable or non-sealable vessel can further comprise at least one rubber gasket and/or plug and/or stopper.

At least one anode can further comprise at least one anode biofilm.

At least one sealable or non-sealable vessel can be configured as at least one of a desk top version that can remain upright when placed upon a horizontal or near horizontal surface, configured in a hanging architecture to be suspending by at least one rigid or semi-rigid and/or flexible element loaded in tension, a side mountable version, or held within a larger container for the purpose of stabilization or for the regulation of environmental conditions such as temperature, light exposure, humidity, etc.

In another embodiment where the invention is operated in an open soil environment, the invention is a substrate-powered energy storage and generation system employing microbial fuel cell technology to power an electrical load, comprising: at least one fuel and/or substrate and/or electrolyte, at least one anode in at least one first location within the fuel and/or substrate and/or electrolyte, at least one means to attach at least one anode wire to at least one anode, at least one cathode in at least one second location, at least one means to attach at least one cathode wire to at least one cathode, a means of routing at least one anode wire and at least one cathode wire to at least one breakout location, and a means of attaching at least one cathode wire to at least one plus terminal and a means of attaching at least one anode wire to at least one minus terminal.

At least one fuel and/or substrate and/or electrolyte can be at least one of soil, mud, river sediment, marine sediment, soil and at least one liquid, garbage and/or bio-degradable garbage, compostable material, microbe and/or bacteria-rich soil, compost starter, soil with vegetation growing within, chemicals, rotting organic and/or inorganic material, human and/or animal waste, guano, minerals, oil and/or oil byproducts, chemical spills on seawater, lake water, and/or water of any location and/or mixture, and/or toxic waste.

The invention can further comprise the ability to be deployed and set up in a location on land and/or water where there has been at least one toxic waste spill and/or release of unwanted chemicals of natural and/or man-made origin.

In another embodiment where the invention is a kit comprising the components necessary to, in conjunction with other materials and/or ingredients externally available, create, facilitate, and/or enhance a substrate-powered energy storage and generation system employing microbial fuel cell technology utilizing a specialized assembly to power an electrical load, comprising packaging to display, house, and/or contain the kit, at least one sealable or non-sealable vessel, a means of enclosing at least one sealable or non-sealable vessel, at least one fuel and/or substrate and/or electrolyte or means to collect same, or instructions as to where to find and how to collect same, at least one anode to be placed in at least one first location within at least one fuel and/or substrate and/or electrolyte within the sealable or non-sealable vessel, at least one anode wire, at least one cathode to be placed in at least one second location within the sealable or non-sealable vessel, at least one cathode wire, at least one minus terminal and at least one plus terminal, at least one gasket and/or stopper and/or plug within which at least one anode wire and/or at least one cathode wire can pass, and at least one electrical load and/or circuit to attach between at least one minus terminal and at least one plus terminal.

The kit packaging can be compostable by shredding and adding to at least one fuel and/or substrate and/or electrolyte either collected and/or received in the kit, thus allowing the packaging to be used in whole or in part to produce electricity.

The kit packaging can further comprise a chemical and/or catalyst to enable and/or facilitate and/or enhance chemical decomposition.

The invention can be styled as, or accompanied by accessories styled as, at least one of a flower, a plush toy, at cartoon and/or real character, an icon, an image, a battery, microbe, bacteria, virus, protista, spore, cell, multi-cellular animal, multi-cellular plant, teddy bear, doll, celebrity, icon, logo, famous place, person, place, and/or thing.

The flower can comprise at least one of at least one root structure, at least one stem, at least one leaf.

At least one stem can be made from at least one of in whole or in part bendable metal, plastic, combinations of metals and/or plastic.

At least one leaf can be made from at least one reflective material and can be positionable to control the direction of reflected and/or radiated light.

The plush toy can be styled as at least one of a microbe, bacteria, virus, protista, spore, cell, multi-cellular animal, multi-cellular plant, teddy bear, doll, celebrity, icon, logo, famous place, person, place, and/or thing.

In another embodiment the invention can be a Mobile Application comprising software to run on at least one smart device to work in connection or association with a substrate-powered energy storage and generation system to acquire data, analyze said data, edit said data, generate new data, and/or display said data and/or said new data.

The invention can further comprise the following steps in no particular order: Taking a video of the substrate-powered energy storage and generation system; Measuring using application software that detects an LED blink frequency using the video camera resident on a smart device, and recognize color and/or light patterns; Process and correlate the blink frequency with power being generated by the substrate-powered energy storage and generation system and correlate this power with estimation of the microbe population necessary to produce the power being generated; Analyze and record at least one value of the power being generated using the “Measure” feature, including the power being generated from different microbial fuel cell (MFC) units to produce the data and/or new data; Display the data and/or new data and/or the power being generated and/or the power being generated from different MFC units in at least one graphic format such that the user can compare power generation and/or growth behavior of at least one substrate-powered energy storage and generation system; Compile the data and/or the new data and/or the power being generated and/or the power being generated from different MFC units in at least one file; Communicate and/or transmit at least one file in the form of at least one of e-mail, wire and/or wireless transmission, upload to at least one server and download from at least one server to at least one user and/or at least one other person; Display via visual presentation and/or audio presentation an educational illustrated comic that tells at least one story of an electric microbe comprising at least one storyline, and at least one storyline being retrievable via a user interaction with at least one button and/or touch screen location on at least one smart device, and at least one storyline comprising at least one in-depth scientific explanation of at least one physical process, thus teaching at least one user about at least one scientific topic; Communicate and/or transmit in the form of at least one of e-mail, wire and/or wireless transmission, upload to at least one server and download from at least one server to at least one user and/or at least one other person at least one supplemental data and/or information comprising at least one of at least one link, common troubleshooting steps, tips and/or recommendations for increasing the MFC power generation.

At least one voltage boosting and/or voltage step-up circuit can comprise at least one of a charge-pump integrated circuit, such as the Seiko S-882Z series as one example shown in FIG. 10, a “Joule-thief” circuit shown in FIG. 9 comprising at least one of at least one capacitor, at least one transistor, at least one ferrite toroid, at least one coiled wire, at least one inductor, and/or at least one LED and/or other load.

In one embodiment of the invention, a kit can also come with an instructional and educational pamphlet and a pair of nitrile gloves. Some kits can include a multimeter and a set of resistors for the user to perform experiments with, such as including but not limited to determining maximum power extractable from a power source by applying different load resistances and measuring the power dissipated by each respective load resistor.

The present invention includes several embodiments of microbial fuel cell (MFC)-powered consumer products, as well as novel electrode and electrode connection designs.

One embodiment of the invention is the novel electrode connections. Connecting an Microbial Fuel Cell (MFC) electrode to an external wire is often a meticulous and expensive process because no reactive metals (copper, tin, zinc, iron, etc.) can be exposed to the harsh preexisting environments or the environment often created by the MFC, such as low pH, chemical byproduct production, etc. For reference, most MFC electrodes are made of high surface area, inert, carbon based materials, such as graphite fiber weaves or felts. Titanium is an inert, malleable, and conductive material that can be used as a wire for electrode connections. However, the titanium must be used alone, with no other metals present, in order to achieve a robust and permanent connection with an electrode. A physical connection between bare titanium (of any form, shape, or size) and the MFC electrode material is necessary to maintain adequate conductivity, the goal being to keep the resistance as low as possible. However significant pressure must be applied and/or significant contact area must exist at the contact point or zone between the titanium and the electrode in order to minimize contact resistance and therefore minimize potential losses resulting from the connection. When current flows through any conductor, conductive element, or connection between conductive elements, the resulting voltage drop is the product of the current and the resistance, and therefore the lower the resistance, the lower the voltage drop. This is often achieved through the use of additional expensive titanium hardware, such as bolts, washers, and nuts. The present invention employs a weave of titanium wire throughout the electrode material, which can be felt, cloth, or other form, as shown in FIG. 1 described in detail herein. The weaving pattern sustains pressure between the titanium wire and the electrode at all times, thus creating a low contact resistance while eliminating the need for additional expensive titanium hardware. The invention includes, but is not limited to, configurations in which the weave forms recognizable patterns such as a face, a flower, an icon, a cartoon character, an image, an embroidered pattern, or any pattern that performs both the electrical connection and has an aesthetic visual appearance. This is a key feature. In some embodiments, the titanium wire is sheathed using an insulating material such as including but not limited to heat shrink tubing, spaghetti tubing and/or non-conductive tubing as it extends from the electrode. In other embodiments, the wire simply pierces the felt and enters the interior of the felt, relying on the contact it has within the interior of the felt, and the longer the length of wire, the greater the surface area and corresponding lower resistance. This has been shown to be an effective way of establishing a connection while minimizing assembly time.

Another embodiment of the invention is an MFC educational kit. In this embodiment the invention consists of a MFC in the design in which there is a container to house the MFC, and this contains an anode, and anode wire, a cathode, a cathode wire, and a plastic lid along with an electronics package connected to the cathode wire and the anode wire of the cell. This embodiment includes, but is not limited to configurations in which the electronics package is physically attached to the top of the MFC. In this embodiment, the electrodes are made of materials including, but not limited to, graphite felt, carbon felt, carbon cloth, graphite cloth, carbon paper, graphite paper, reticulated carbon foam, and/or reticulated graphite foam, and the electrode connection wire can be composed of sheathed or unsheathed titanium or other metal listed previously. The electrodes can be soaked in water or other solutions to limit the release of loose electrode fibers into the air, though the invention is not limited to nor does it require this construction step. The fuel for the MFC can consist of soil and water, and can be placed into the jar so that the anode is buried within the fuel, which is not an oxygen rich environment. The cathode can rest on top of the soil and water, physically contacting this fuel, yet being sufficiently exposed to the air above it to utilize this naturally occurring oxygen-rich environment. In this configuration, the user is able to input the fuel of the user's choosing into the jar via the top opening, after temporarily removing the cathode. The wires protruding from the electrodes can be curled or coiled in such a fashion as to enable the user to easily set the height of each electrode within the container, though the invention is not limited to this strategy.

The invention includes a variety of electronics packages such as including but not limited to that described above, and this can provide a means for the user to interact with a MFC in an entertaining and/or educational way. In one embodiment, the electronics package can include the use of an LED light or other light source or other sensory device which is powered constantly or intermittently through the use of a voltage step up charge-pump circuit, at least one capacitor, and/or “Joule-thief” circuits consisting of transistors, resistors, and ferrite toroid core and/or inductive components, for example. These circuits are particularly useful for this application given the low voltages (0-1.2V) created by the MFC, which can require the need for voltage step-up circuitry to power useful loads and/or devices. The blinking of an LED or other light-producing element is a visual indication that the MFC is producing power, and the frequency or the brightness of the blinking can correspond to the actual power being produced by the MFC through a characterized relationship. Other indications can be audio circuits producing sound, radio receivers, mechanical motion, other sensory outputs and/or any combination.

In some embodiments, there is an accompanying mobile application that enables mobile devices or smart devices to detect the brightness or blink frequency of the MFC's LED or other light-producing element and correlate that to a power generation value and/or a microbial population estimation through a characterized relationship.

In some embodiments, the invention can include small electronics devices to be powered by the MFC, such as a clock, thermometer, buzzer, etc.

In some embodiments, the MFC is connected to a sensor system that collects environmental data such as temperature, soil moisture, chemical composition, pressure, light level and/or spectrum, radiation, etc. and transmits that data to a user using Bluetooth low energy modules or other means that can be both wireless and physically connected using wires and/or optical links.

In some embodiments, the electronics can be designed to allow the user to plug in his own components for experimentation, such other LEDs, capacitors, or small electronics packages or kits or any construction and/or circuit that can run on the voltage and current provided by one or more MFCs either alone or in any series and/or parallel configuration.

In some embodiments the electronics package can include a series of resistors that the user may turn on or off using a switch, such as the DIP switch shown in FIG. 5, so as to place a switchable resistance in series with a load, thus simulating a potentiometer in series with a load using the MFC as the power source. The LED or other sensory device or load would be in series with the resistors and connected between the electrode wires protruding from the MFC when switched on. Different combinations of switches could produce different parallel combinations, producing many different resistance values in series with the load, which in this example happens to be an LED.

In some embodiments of this kit, the packaging material can be torn up into pieces and placed within the MFC unit, to act as a fuel source for the MFC system.

In another embodiment an MFC can be configured to look like a plant or flora. The invention includes but is not limited to configurations in which the anode resembles the roots of the plant. The anode can be made of any suitable electrode material, such as graphite felt, graphite, carbon felt, graphite felt, carbon cloth, graphite cloth, carbon paper, graphite paper, reticulated carbon foam, and/or reticulated graphite foam as examples, and can be configured in any form and size. The anode can employ any form of connection such as the woven, threaded, or penetrating wire connection described herein. In order for the MFC to function, this root-like anode may be buried or potted within some body of soil or other medium or combination of media to act as a substrate. This invention includes, but is not limited to embodiments wherein the cathode resembles some other component of a plant, such as leaves. The cathode can be made of any suitable material such as graphite felt or cloth, for example, and can be configured in any form and size, and can employ any form of connection such as the woven, threaded, or penetrating wire connection described herein.

In some embodiments, the plant-like MFC would be but is not limited to being potted in a planter or container much like a living plant.

In some embodiments, the plant-like MFC would be potted in open soil, or within some other substrate, without the use of a containment vessel.

Designing the MFC to resemble a plant can create opportunities for a wide variety of electronic products to be developed for consumer use. One embodiment is to configure the plant to perform the function of at least one desk lamp that is aesthetically pleasing. A small light including, but not limited to at least one LED can be powered in the center of at least one flower-like structure. The stem of the plant can transmit all the required power from the MFC to the light-producing components.

In other embodiments, the stem can be made of at least one of in whole or in part bendable metal, plastic, combinations of metals and/or plastic. This enables the user to move and position light where light is needed. In any configurations mentioned thus far, at least one light source can emit broadly so as to create ambiance, to emit narrowly so as to illuminate certain a certain area or areas of interest to the user, and the light can be emitted in any profile between maximum and minimum brightness throughout the entire light-radiating spatial angles. This invention includes, but is not limited to configurations in which the petals of any flower on the plant are made of a reflective material, so that the light is reflected to certain range of angles, so as to maximize illumination of at least one area of interest to the user.

The invention can include all types of electronic packages. In some embodiments, the MFC can charge at least one capacitor and/or at least one battery, with or without a voltage step-up circuit to produce voltage capable of performing useful work upon demand by or control of the user. This can include but is not limited to, configurations in which the power is used switched or controlled by the user to power at least one LED or other light source. The light source can change colors over time, and this can include LED of different colors or three color LEDs or two color LEDs or any combination to indicate to the user when the stored charge is depleting, rate of depletion, charging, rate of charge, voltage, though the invention is not limited to indication of these parameters.

In some embodiments, the electronics can be contained within at least one enclosure that is consistent with the aesthetic of the MFC.

MFC technology is ideal for applications requiring low power. In another embodiment the invention can be used as a night light and/or as mood lighting for both residential and commercial spaces. One example incorporates MFC ambient lighting systems discreetly in everyday environments by using plant life to conceal the use of the electronics generating the light. In some embodiments, the lighting hardware can be placed beneath the container of the MFC. The fuel for this MFC, as with any MFC design described herein, can consist of any substrate. In this embodiment soil is used in as the fuel, with vegetation growing within it. The actual configuration/architecture/materials employed in this MFC can reflect any configuration/architecture/materials outlined herein.

In other embodiments, MFC technology can be styled as, or with an accompanying accessory styled as, an electric bacteria plush toy or item. MFC kits can be used for educational purposes, and the micro-organisms that drive the MFC system can be a focal point for teaching about biological, chemical, and physical processes. In this embodiment, the invention can be or include a plush toy representing the microbes with an MFC system, particularly but not limited to the Shewanella and Geobacter bacteria. In some embodiments, the Pili of this plush toy can light up with light as to show electron or energy flow through them. However, this light-up action is not limited to the Pili and can be used on various places on or within the plush toy.

Although preferred embodiments of the present invention have been described herein it will be understood by those skilled in the art that the present invention should not be limited to the described preferred embodiments. Rather, various changes and modifications can be made within the spirit and scope of the present invention.

All of the material in this patent document issue subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BRIEF DESCRIPTION OF THE SEVERAL VIEWS OF THE DRAWING

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIGS. 1A and 1B show typical cross-sectional views of an electrode with wire connections.

FIG. 2 shows a cross-sectional view of an MFC kit with the components of the energy generation system.

FIG. 3 shows an MFC energy generation system with an electronics package in communication with a smart device running an application.

FIG. 4 shows the chemical environment that produces a low voltage differential, a voltage step-up circuit, and an electrical load.

FIG. 5 shows a voltage source providing voltage to an array of four dip switches, each switch in series with a resistor, in series with an LED.

FIG. 6 shows an MFC configured to look like a flower in a flower pot.

FIG. 7 shows an MFC configured as a hanging night light or mood light.

FIG. 8 shows an accompanying accessory plush toy styled as a microbe.

FIG. 9 shows an embodiment of a “Joule-thief” circuit used for an MFC system to power a load.

FIG. 10 shows an embodiment of a charge-pump circuit used for an MFC system to power a load.

### DETAILED DESCRIPTION OF THE INVENTION

Detailed descriptions of particular embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

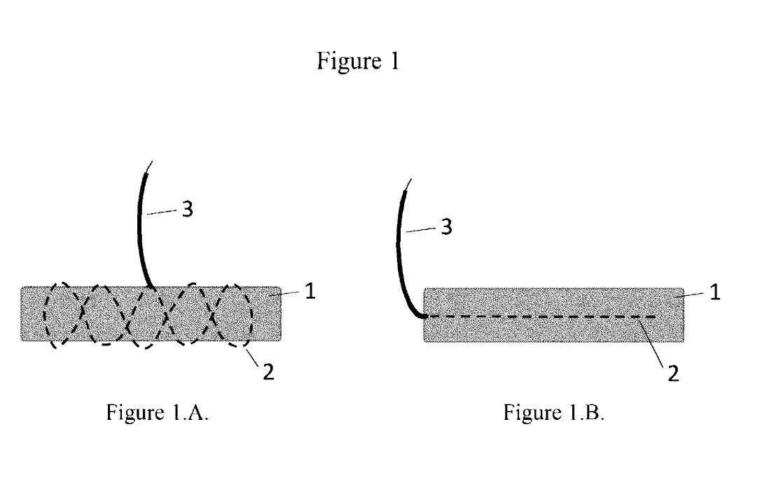

FIGS. 1A and 1B show typical cross-sectional views of an electrode. Typical electrode 1 can be made from graphite, graphite felt, carbon felt, carbon cloth, graphite cloth, carbon paper, graphite paper, reticulated carbon foam, and/or reticulated graphite foam. Titanium wire 3 forms the titanium wire woven within and/or inserted into electrode 2, and this increases the surface area of the bare non-reactive titanium wire 3 which contacts the higher resistance electrode 1, thus lowering the contact resistance between the two. The lower the resistance of this union of two materials, the lower the voltage drop and corresponding loss at the source of power itself. Resistance multiplied by current equals voltage, and this voltage is the voltage drop that lowers deliverable power and efficiency of any power transmission system.

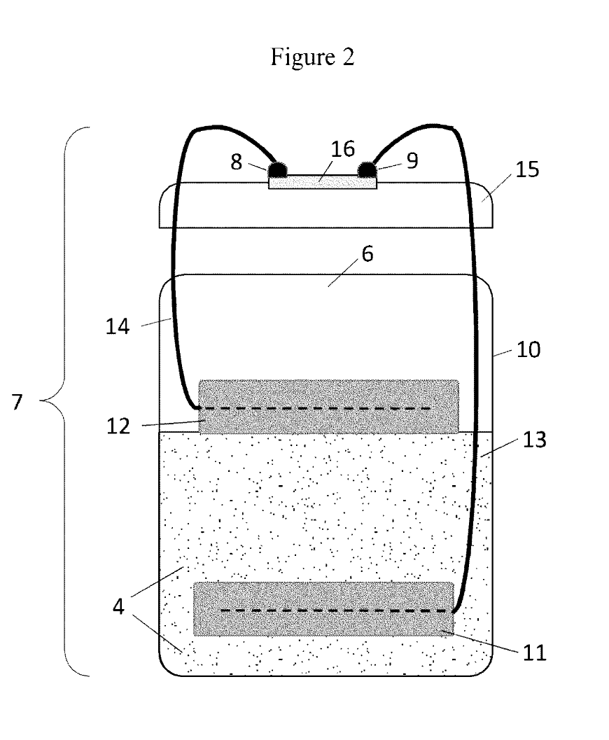

FIG. 2 shows a cross-sectional view of an MFC kit 7 with the components of the energy generation system. The container plastic jar 10 houses the anode graphite felt electrode 11 which is located within the fuel and/or substrate and/or electrolyte 4. This creates an oxygen poor environment which ultimately produces the lower redox potential source needed to create a voltage differential between the two electrodes. The cathode graphite felt electrode 12 is located on top of the fuel and/or substrate and/or electrolyte 4, and exposed to the oxygen-rich air 6 which ultimately produces the higher redox potential source needed to create a voltage differential between the two electrodes. Connected to the anode graphite felt electrode 11 is the sheathed titanium anode wire 13, and this passes through the fuel and/or substrate and/or electrolyte 4, through the air 6 within the interior of the container plastic jar 10, through the screw-on or snap-fit plastic lid 15, and terminates at the negative terminal 9. Connected to the cathode graphite felt electrode 12 is the sheathed titanium cathode wire 14, and this passes through the air 6 within the interior of the container plastic jar 10, through the screw-on or snap-fit plastic lid 15, through the rubber stopper 18, and terminates at the positive terminal 8. The electronics package 16 is connected between the positive terminal 8 and negative terminal 9.

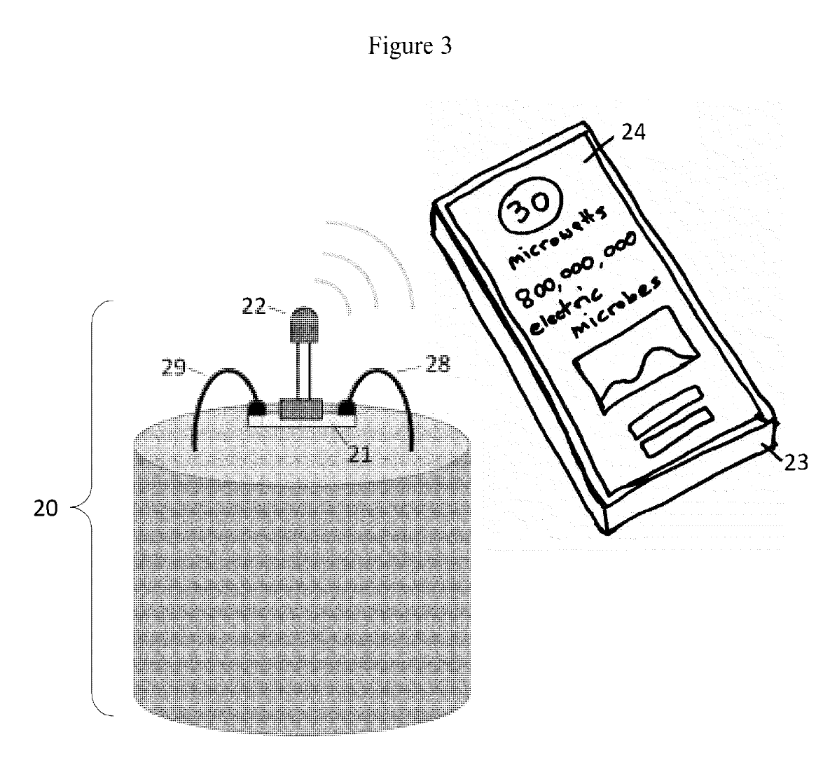

FIG. 3 shows an MFC energy generation system 20 with an electronics package in communication with a smart device 23 running a mobile application and display 24. Shown is the cathode wire 29 and anode wire 28, which feeds in this embodiment a LED blinker circuit module 22. In this embodiment, the LED blinking frequency is dependent on the power output and/or microbial population of the MFC energy generation system 20. In this embodiment, the smart device 23 detects the LED blink frequency, calculates the power output and/or microbial population of the MFC energy generation system 20, and displays that information with a graphical display 24. Other embodiments can utilize other wireless communication subsystem 21 such as radio frequency or Bluetooth modules to communicate information to the smart device 23. The electronics package can be any circuit including a voltage step-up, charge-pump, or “Joule-thief” circuit to produce the higher voltages that are required for some circuits and applications.

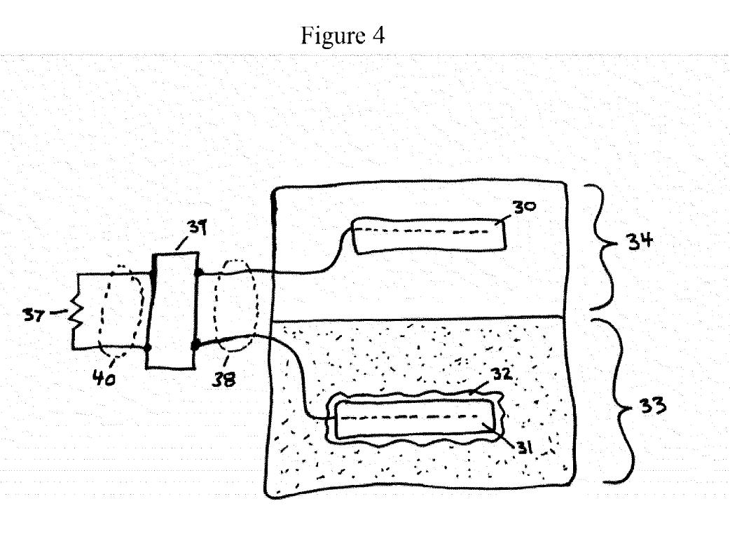

FIG. 4 shows the chemical environment that produces a low voltage differential, a voltage step-up circuit, and an electrical load. Cathode 30 is contained and/or located within a region with high redox potential 34, which typically would be an oxygen-rich environment such as might be present in exposure to the air. Anode 31 is contained and/or located within a region with low redox potential 33, such as being buried within soil or another fuel and/or substrate. There can also be an anode microbial biofilm 32 surrounding the anode 31. Cathode 30 and anode 31 produce a low voltage directly from chemical potential differential 38, which feeds voltage step-up, charge-pump, or “Joule-thief” circuit 39, which produces higher voltage output 40 to feed a useful load 37 requiring high voltage output. This load can be any number of devices including sensors, light producing elements, sound producing elements, mechanical motion producing devices, electromagnetic radiation producing devices employing modulation of any kind, information processing systems, and or circuits of any kind.

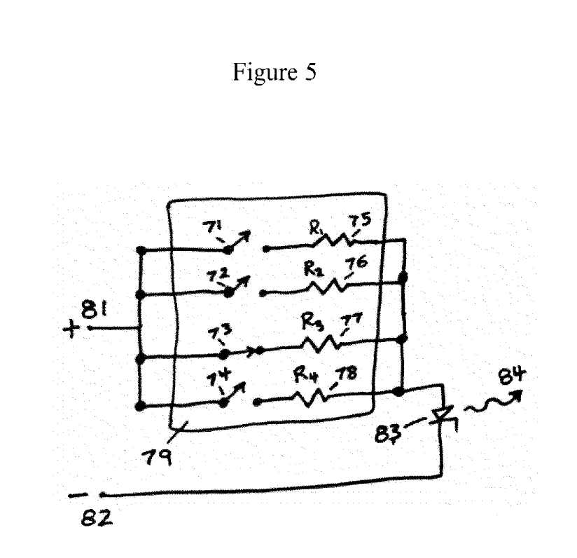

FIG. 5 shows a voltage source providing voltage to an array of four dip switches, each switch in series with a resistor, in series with an LED, thus effectively producing the effect of a simulated rheostat resulting from selecting different combinations of switches and corresponding resistors. Shown is + voltage source 81 and − voltage source 82 which feeds the four dip switch package 79, the resistors, and LED 83. Switch one 71 can turn on or off R1 75, though in this case switch one 71 is shown on the open position, switch two 72 can turn on or off R2 76, though in this case switch two 72 is shown on the open position, switch three 73 can turn on or off R3 77, though in this case switch three 73 is shown on the closed position, and switch four 74 can turn on or off R4 78, though in this case switch four 74 is shown on the open position. In this example, only switch three 73 is closed, thus placing R3 77 in series with LED 83 which produces light 84. If hypothetically, switch one 71 were also closed, R1 75 would now be in parallel with R3 77, and the resulting resistance would be a classical resistance product over sum relationship, thus

Rtotal=(R1 75)(R3 77)/((R1 75)+(R3 77)).

With four dip switches there are sixteen possible combinations of switches.

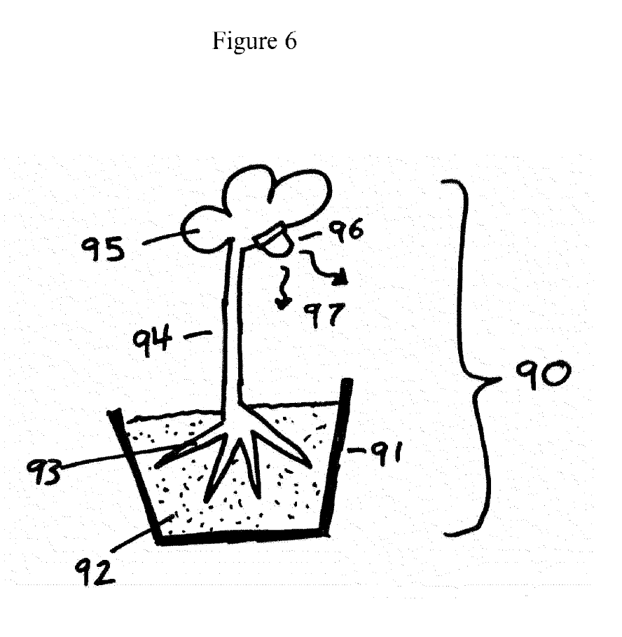

FIG. 6 shows an MFC configured to look like a flower-like structure 90 in a flower pot 91. In this embodiment the soil or other substrate 92 is in the flower pot 91. Within the soil or other substrate 92 is a root anode 93 which is attached to stem structure 94. The cathode leaves and/or petals 95 are also attached to the stem structure 94. At least one LED light 96 can be positionable to focus light 97 in the desired direction.

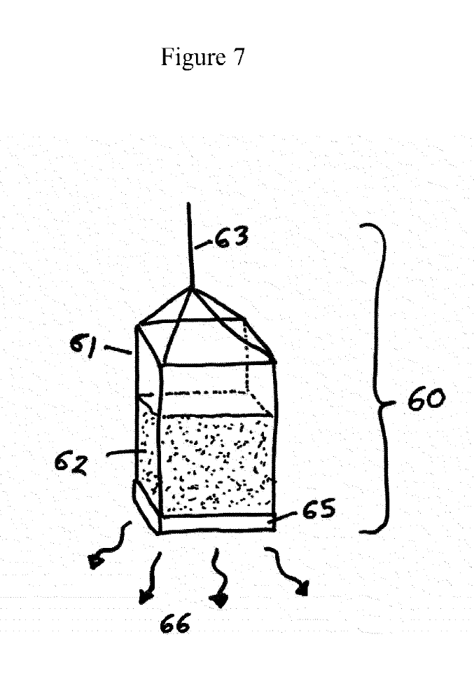

FIG. 7 shows an MFC configured as a hanging night light or mood light 60. In this configuration, the container 61 containing substrate 62 is suspended from above via the support line 63. All the electrical other components mentioned are contained within this structure and it is unnecessary for clarity to re-explain them. The light producing element 65 is located on the bottom of the container 61, and produces light 66 which can be directed outward and/or downward in either a narrow beam or wide beam. The light can be of any color and/or color combination, can incorporate dimming and/or fading and/or color changing features in whole or in part, together or individually, and the light can, though not shown, be turned on and off via wireless communication.

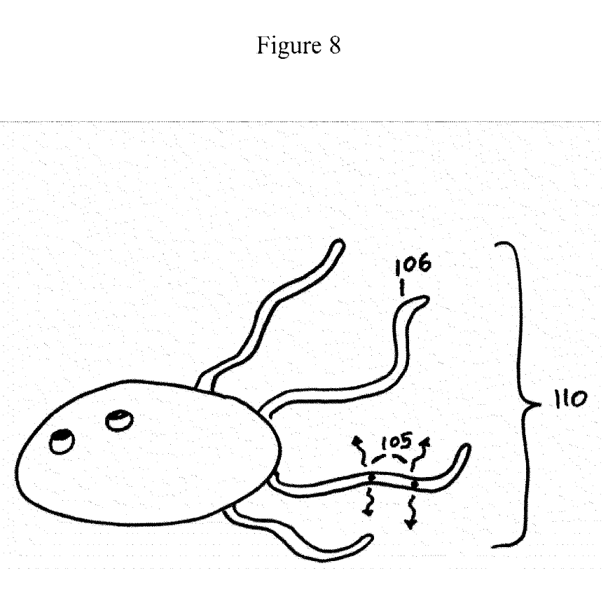

FIG. 8 shows an MFC configured as a plush toy styled as a microbe. The Pili 106 of this plush toy styled as a microbe 110 are shown lighting up with light 105 as if to show electron or energy flow through them. However, this light-up action is not limited to the Pili and can be used on various places on or within the plush toy styled as a microbe 110. This embodiment is not limited to representing a microbe and can be any type of toy which can employ the power generating principle of an MFC, or accompany a MFC energy generation system.

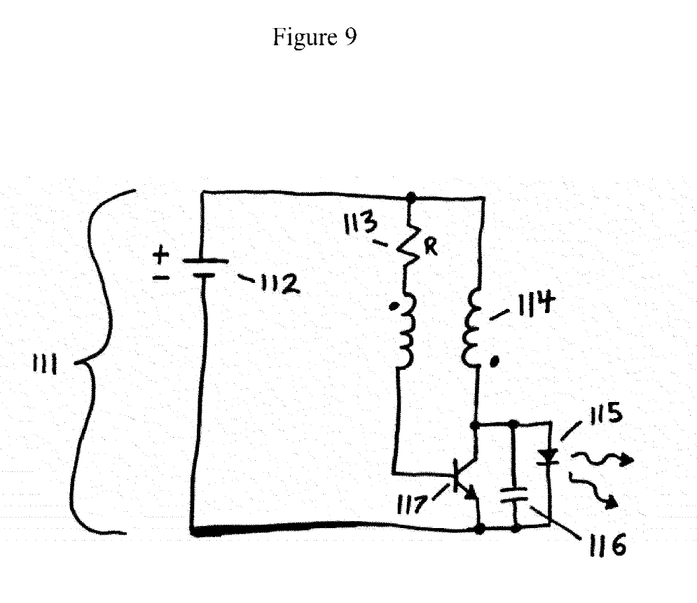

FIG. 9 shows an example of a “Joule-thief” circuit 118 which can be used to step-up the voltage coming from the MFC energy generation system 112 into a voltage usable by a given load. In this embodiment, the circuit employs the MFC energy generation system 112, a resistor 113, an inductor or ferrite toroid with wire coil 114, a transistor 117, an LED light 115 and capacitor 116.

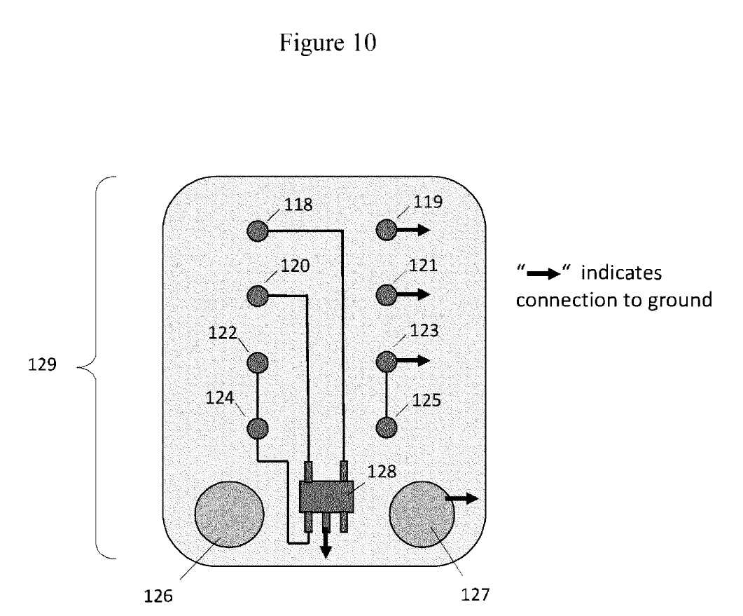

FIG. 10 shows an example circuit design for an MFC energy generating system to employ a charge-pump integrated circuit (IC) 128, such as the Seiko S-882Z series, to power a load. In this embodiment the MFC system's cathode wire is connected to positive terminus 118, which is connected to the power supply input pin of the charge-pump IC, and the MFC system's anode wire is connected to negative terminus 119, which is connected to ground. The positive lead of a capacitor is connected to connection point 120, which is connected to the startup connection point of the charge-pump IC, and the negative lead of the capacitor is connected to ground via connection point 121. The load's positive lead is connected to connection point 124, which is connected to the output pin of the charge-pump IC, and the load's negative lead is connected to ground via connection point 125. If needed, a secondary capacitor can be connected in parallel to the load by connecting the capacitor's positive lead to connection point 122, which is connected to connection point 124, and the capacitor's negative lead to ground via connection point 125. If needed, two bare pads can be included in the circuit for easy connection to the positive terminus 126 and ground 127. This is useful for checking the voltage of the system with a multimeter without having to disconnect anything.

### Component List for Drawings

Following is a partial list of the components depicted in the drawings:

| Component | |
|---|---|
| Number | Component Description |
| 1 | typical electrode |
| 2 | titanium wire woven within electrode |
| 3 | titanium wire |
| 4 | fuel and/or substrate and/or electrolyte |
| 6 | air |
| 7 | MFC kit |
| 8 | positive terminal |
| 9 | negative terminal |
| 10 | container plastic jar |
| 11 | anode graphite felt electrode |
| 12 | cathode graphite felt electrode |
| 13 | sheathed titanium anode wire |
| 14 | sheathed titanium cathode wire |
| 15 | screw-on or snap-fit plastic lid |
| 16 | electronics package |
| 20 | MFC energy generation system |
| 21 | wireless communication subsystem |
| 22 | blinking LED module |
| 23 | smart device |
| 24 | mobile application and display |
| 28 | anode wire |
| 29 | cathode wire |
| 30 | cathode |
| 31 | anode |
| 32 | anode biofilm |
| 33 | region with low redox potential |
| 34 | region with high redox potential |
| 37 | useful load requiring high voltage output |
| 38 | low voltage directly from chemical potential differential |
| 39 | voltage step-up, charge-pump, and/or “Joule-thief” circuit |
| 40 | high voltage output |
| 60 | hanging night or mood light |
| 61 | container |
| 62 | substrate |
| 63 | support line |
| 65 | light producing element |
| 66 | light |
| 71 | switch one |
| 72 | switch two |
| 73 | switch three |
| 74 | switch four |
| 75 | R1 |
| 76 | R2 |
| 77 | R3 |
| 78 | R4 |
| 79 | four dip switch package |
| 81 | +voltage source |
| 82 | −voltage source |
| 83 | LED |
| 84 | light |
| 90 | flower-like structure |
| 91 | flower pot |
| 92 | soil or other substrate |
| 93 | root anode |
| 94 | stem structure |
| 95 | cathode leaves |
| 96 | at least one LED light |
| 97 | light |
| 105 | light |
| 106 | Pili |
| 110 | plush toy styled as a microbe |
| 111 | Example “Joule-thief” circuit |
| 112 | MFC energy generation system + and − leads. |
| 113 | Resistor |
| 114 | Ferrite Toroid or inductor |
| 115 | LED |
| 116 | capacitor |
| 117 | Transistor |
| 118 | positive terminus for cathode wire |
| 119 | negative terminus for anode wire |
| 120 | connection point for capacitor's positive lead |
| 121 | connection point for capacitor's negative lead |
| 122 | connection point for secondary capacitor's positive lead |
| 123 | connection point for secondary capacitor's negative lead |
| 124 | connection point for load's positive lead |
| 125 | connection point for load's negative lead |
| 126 | bare pad for easily contacting positive terminus for the |
| | cathode wire |
| 127 | bare pad for easily contacting ground for the anode wire |
| 128 | charge-pump integrated circuit |
| 129 | example circuit diagram for MFC system employing |
| | charge-pump circuit to power a load |

### DEFINITIONS

These definitions are in addition to the words and phrases specifically defined in the body of this application.

MFC is Microbial fuel cell (MFC) technology, which employs microorganisms to generate electromotive potential which can cause the flow of current in a circuit.

Substrate is a surface or material on or from which an organism lives, grows, and/or obtains its nourishment. This can be a fuel which can include soil, mud, soil and at least one liquid, garbage and/or bio-degradable garbage, compostable material, microbe and/or bacteria-rich soil, soil with vegetation growing within, chemicals, rotting organic and/or inorganic material, human and/or animal waste, guano, minerals, oil and/or oil byproducts, chemical spills on seawater, lake water, and/or water of any mixture, and/or toxic waste.

Smart device: In the context of this invention a smart device is a mobile phone and/or what is commonly referred to as a smart phone such as including but not limited to an iPhone or an Android phone, a laptop computer, a desktop computer, a mainframe computer, a tablet, a reader, a projection device and/or system. The smart device can further comprise at least one of at least one camera, video camera, RFID, GPS circuitry, at least one communication receiver device and/or the at least one communication transmitter device and/or the at least one communication transmitter/receiver device employing at least one of Wi-Fi, radio wave and/or electromagnetic radiation, and/or Bluetooth.

The term “his” is the convenient possessive form and is in no way meant to imply gender. “His” can refer to the user or any other entity that can be a person.

As used herein, the term “and/or,” when used in a list of two or more items, means that any one of the listed items can be employed by itself, or any combination of two or more of the listed items can be employed. For example, if a device is described as containing components A, B, and/or C, the composition can contain A alone; B alone; C alone; A and B in combination; A and C in combination; B and C in combination; or A, B, and C in combination.

What is claimed:

1. A substrate-powered energy storage and generation system employing microbial fuel cell technology utilizing a specialized assembly to power at least one electrical load, comprising:
at least one sealable or non-sealable vessel;
a means of enclosing said at least one sealable vessel;
at least one fuel and/or substrate and/or electrolyte or means to collect same, or instructions as to where to find and how to collect same, within or to be placed within, said sealable or non-sealable vessel;
at least one anode in at least one first location within said fuel and/or substrate and/or electrolyte within said sealable or non-sealable vessel;
at least one means to attach at least one anode wire to said at least one anode;
at least one cathode in at least one second location within said sealable or non-sealable vessel;
at least one means to attach at least one cathode wire to said at least one cathode;
a means of passing said at least one anode wire and said at least one cathode wire through said means of enclosing said at least one sealable or non-sealable vessel;
a means of attaching said at least one cathode wire to at least one plus terminal and a means of attaching said at least one anode wire to at least one minus terminal.

2. The substrate-powered energy storage and generation system of claim 1 further comprising at least one electrical load placed between said at least one plus terminal and said at least one minus terminal.

3. The substrate-powered energy storage and generation system of claim 1 wherein said at least one anode and said at least one cathode exhibit at least one different redox potential.

4. The substrate-powered energy storage and generation system of claim 3 wherein said at least one anode is placed in a nutrient-rich oxygen-poor environment and said at least one cathode is placed within an oxygen-rich environment.

5. The substrate-powered energy storage and generation system of claim 4 wherein said oxygen-poor environment either includes at least one of Shewanella bacteria, Deltaproteobacteria, Geobacter bacteria, Clostridia bacteria and/or at least one other bacteria and/or is created, facilitated, and/or enriched by the addition of at least one of Shewanella bacteria, Deltaproteobacteria, Geobacter bacteria, Clostridia bacteria and/or at least one other bacteria.

6. The substrate-powered energy storage and generation system of claim 4 wherein said oxygen-poor environment is located at at least one depth under soil and/or said at least one fuel and/or substrate and/or electrolyte and said oxygen-rich environment is positioned in at least one location exposed to the air and or on top of said soil and/or at least one substrate.

7. The substrate-powered energy storage and generation system of claim 2 wherein said electrical load is at least one of at least one LED, desk lamp, night light, mood lighting, clock, thermometer, moisture sensor, temperature sensor, pressure sensor, sound sensor, other sensor or combination of sensors, buzzer, audio circuit, radio transmitter and/or receiver, Bluetooth transmitter and/or receiver, a capacitor, a resistor, an active circuit, a rechargeable battery, voltage step-up circuit and/or “Joule-thief” circuit acting as an intermediary sub-system interposed between said substrate-powered energy storage and generation system of claim 1 and said at least one electrical load.

8. The substrate-powered energy storage and generation system of claim 1 wherein said at least one fuel and/or substrate and/or electrolyte is at least one of soil, mud, soil and at least one liquid, garbage and/or bio-degradable garbage, compostable material, microbe and/or bacteria-rich soil, soil with vegetation growing within, chemicals, rotting organic and/or inorganic material, human and/or animal waste, guano, minerals, oil and/or oil byproducts, chemical spills on seawater, lake water, and/or water of any mixture, and/or toxic waste.

9. The substrate-powered energy storage and generation system of claim 1 wherein said at least one anode wire and at least one cathode wire is at least one of titanium, a titanium alloy, a non-reactive conductor, conductive ink, gold, copper, tin, aluminum, steel, stainless steel, and/or other conductive alloy, mixture, suspension, material, or substance.

10. The substrate-powered energy storage and generation system of claim 1 wherein said at least one anode and at least one cathode is made from at least one of graphite, carbon felt, graphite felt, carbon cloth, graphite cloth, carbon paper, graphite paper, reticulated carbon foam, reticulated graphite foam, aluminum, steel, and/or stainless steel.

11. The substrate-powered energy storage and generation system of claim 10 wherein said at least one means to attach at least one anode wire to said at least one anode is by weaving, threading, and/or penetrating at least one titanium wire through said at least one anode in at least one place and said at least one means to attach at least one cathode wire to said at least one cathode is weaving, threading and/or at least one titanium wire through said at least one cathode in at least one place.

12. The substrate-powered energy storage and generation system of claim 1 further comprising at least one voltage boosting, voltage step-up, and/or “Joule-thief” circuit to produce at least one voltage that is higher than the voltage produced between said at least one anode and said at least one cathode and/or downloadable Mobile Application comprising software to run on at least one smart device to work in connection or association with the said substrate-powered energy storage and generation system to acquire data, analyze said data, generate new data, and/or display said data and/or said new data, wherein said at least one voltage boosting and/or voltage step-up circuit comprises at least one of a charge-pump integrated circuit, such as the Seiko S-882Z integrated circuit series, and/or a “Joule-thief” circuit comprising at least one of at least one capacitor, at least one transistor, at least one ferrite toroid, at least one coiled wire, at least one inductor, and/or at least one LED and/or other load.

13. The substrate-powered energy storage and generation system of claim 1 further comprising a low power circuit that either produces at least one wireless transmission of information comprising at least one parameter and/or produces a blinking LED representative of at least one attribute, said at least one attribute being a function of at least one parameter, wherein said at least one attribute is at least one of:
LED on time;
LED off time;
LED brightness during said LED on time;
LED frequency and period, wherein said frequency is the mathematical reciprocal of said period, and said period is the mathematical sum of said LED on time and said LED off time;
and wherein said at least one parameter is at least one of:
open cell voltage under no load;
voltage under load;
voltage increase and/or voltage decrease;
current delivered to at least one load;
power delivered to at least one load.

14. The substrate-powered energy storage and generation system of claim 13 further comprising an application comprising software to run on at least one smart device, using at least one capability of said at least one smart device, said application working in conjunction with and supplemental to said substrate-powered energy storage and generation system to provide at least one status indication and/or indication of said at least one parameter and/or said at least one attribute and/or to store information and to make accessible and display at least one of the following: said at least one parameter, said at least one parameter as a function of time, the mathematical integral of said at least one parameter as a function of time between at least one first time and at least one second time, estimated microbial population contributing to power generation, other information associated with said substrate-powered energy storage and generation system.

15. The substrate-powered energy storage and generation system of claim 1 wherein said means of passing said at least one anode wire and said at least one cathode wire through said means of enclosing said at least one sealable or non-sealable vessel further comprises at least one rubber gasket and/or plug and/or stopper.

16. The substrate-powered energy storage and generation system of claim 1 wherein said at least one anode further comprises at least one anode biofilm.

17. The substrate-powered energy storage and generation system of claim 1 wherein said at least one sealable or non-sealable vessel is configured as at least one of a desk top version that remains upright when placed upon a horizontal or near horizontal surface, configured in a hanging architecture to be suspending by at least one rigid or semi-rigid and/or flexible element loaded in tension, a side mountable version, or held within a larger container for the purpose of mechanical stabilization and/or for the regulation and/or isolation of environmental conditions including but not limited to temperature, chemical composition and/or chemical exchange with the ambient environment, light exposure, humidity.
In Open Soil

18. A substrate-powered energy storage and generation system employing microbial fuel cell technology to power an electrical load, comprising:
at least one fuel and/or substrate and/or electrolyte;
at least one anode placed in at least one first location within said fuel and/or substrate and/or electrolyte;
at least one means to attach at least one anode wire to said at least one anode;
at least one cathode placed in at least one second location;
at least one means to attach at least one cathode wire to said at least one cathode;
a means of routing said at least one anode wire and said at least one cathode wire to at least one breakout location;
a means of attaching said at least one cathode wire to at least one plus terminal and a means of attaching said at least one anode wire to at least one minus terminal.

19. The substrate-powered energy storage and generation system of claim 18 wherein said at least one fuel and/or substrate and/or electrolyte is at least one of soil, mud, soil and at least one liquid, garbage and/or bio-degradable garbage, compostable material, compost starter, microbe and/or bacteria-rich soil, soil with vegetation growing within, chemicals, rotting organic and/or inorganic material, human and/or animal waste, guano, minerals, oil and/or oil byproducts, chemical spills on seawater, lake water, and/or water of any location and/or mixture, and/or toxic waste.

20. The substrate-powered energy storage and generation system of claim 19 further comprising the ability to be deployed and set up in a location on land and/or water where there has been at least one toxic waste spill and/or release of unwanted chemicals of natural and/or man-made origin.
Kit

21. A kit comprising the components necessary to, in conjunction with other materials and/or ingredients externally available, to create, facilitate, and/or enhance a substrate-powered energy storage and generation system, employing microbial fuel cell technology utilizing a specialized assembly to power an electrical load, comprising:
packaging to display, house, and/or contain said kit;
at least one sealable or non-sealable vessel;
a means of enclosing said at least one sealable or non-sealable vessel;
at least one fuel and/or substrate and/or electrolyte or means to collect same, or instructions as to where to find and how to collect same;
at least one anode to be placed in at least one first location within said at least one fuel and/or substrate and/or electrolyte within said sealable or non-sealable vessel;
at least one anode wire;
at least one cathode to be placed in at least one second location within said sealable or non-sealable vessel;
at least one cathode wire;
at least one minus terminal and at least one plus terminal;
at least one electrical load and/or circuit to attach between said at least one minus terminal and at least one plus terminal.

22. The kit of claim 21 wherein said packaging is compostable by shredding and adding to said at least one fuel and/or substrate and/or electrolyte either collected and/or received in said kit, thus allowing said packaging to be used in whole or in part to produce electricity.

23. The kit of claim 22 wherein said packaging further comprises a chemical and/or catalyst to enable and/or facilitate and/or enhance chemical decomposition.

24. The substrate-powered energy storage and generation system of claim 21 wherein at least one of:
a. said electrical load is at least one of at least one LED, desk lamp, night light, mood lighting, clock, thermometer, moisture sensor, temperature sensor, pressure sensor, sound sensor, other sensor or combination of sensors, buzzer, audio circuit, radio transmitter and/or receiver, Bluetooth transmitter and/or receiver, a capacitor, a resistor, an active circuit, a rechargeable battery, voltage step-up circuit and/or “Joule-thief” circuit acting as an intermediary sub-system interposed between said substrate-powered energy storage and generation system of claim 1 and said at least one electrical load;
b. the invention further comprising at least one voltage boosting, voltage step-up circuit, and/or “Joule-thief” circuit to produce at least one voltage that is higher than the voltage produced between said at least one anode and said at least one cathode and/or downloadable Mobile Application comprising software to run on at least one smart device to work in connection or association with the said substrate-powered energy storage and generation system to acquire data, analyze said data, generate new data, and/or display said data and/or said new data; and/or
c. the invention further comprising a low power circuit that either produces at least one wireless transmission of information comprising at least one parameter and/or produces a blinking LED representative of at least one attribute, said at least one attribute being a function of at least one parameter, wherein said at least one attribute is at least one of:
LED on time;
LED off time;
LED brightness during said LED on time;
LED frequency and period, wherein said frequency is the mathematical reciprocal of said period, and said period is the mathematical sum of said LED on time and said LED off time;
and wherein said at least one parameter is at least one of:
open cell voltage under no load;
voltage under load;
voltage increase and/or voltage decrease;
current delivered to at least one load;
power delivered to at least one load;
and/or further comprising an application comprising software to run on at least one smart device, using at least one capability of said at least one smart device, said application working in conjunction with and supplemental to said substrate-powered energy storage and generation system to provide at least one status indication and/or indication of said at least one parameter and/or said at least one attribute and/or to store information and to make accessible and display at least one of the following: said at least one parameter, said at least one parameter as a function of time, the mathematical integral of said at least one parameter as a function of time between at least one first time and at least one second time, estimated microbial population contributing to power generation, other information associated with said substrate-powered energy storage and generation system.
Flower

25. The substrate-powered energy storage and generation system of claim 24 wherein said flower comprises at least one of at least one root structure, at least one stem, at least one leaf.

26. The substrate-powered energy storage and generation system of claim 25 wherein said at least one stem is made from at least one of in whole or in part bendable metal, plastic, combinations of metals and/or plastic.

27. The substrate-powered energy storage and generation system of claim 25 wherein said at least one leaf is made from at least one reflective material and is positionable to control the direction of reflected and/or radiated light.

28. The substrate-powered energy storage and generation system of claim 21 wherein the invention is visually styled or modeled as at least one of a flower, a toy, a cartoon and/or real character, an icon, an image, a battery, microbe, bacteria, virus, protista, spore, cell, multi-cellular animal, multi-cellular plant, celebrity, logo, famous place, person, place, and/or thing and/or accompanied by at least one toy, a plush toy, a character or figurine, teddy bear, and/or doll, and said plush toy is styled as at least one of a microbe, bacteria, virus, protista, spore, cell, multi-cellular animal, multi-cellular plant, teddy bear, doll, celebrity, icon, logo, famous place, person, place, and/or thing.

29. A Mobile Application comprising software to run on at least one smart device to work in connection or association with a substrate-powered energy storage and generation system to acquire data, analyze said data, generate new data, and/or display said data and/or said new data.

30. The invention of claim 29 further comprising the following steps in no particular order:
Taking a video of said substrate-powered energy storage and generation system;
Measuring using application software that detects an LED blink frequency using the video camera resident on said smart device, and recognize color and/or light patterns;
Process and correlate said blink frequency with power being generated by said substrate-powered energy storage and generation system and correlate said power with estimation of the microbe population necessary to produce said power being generated;
Analyze and record at least one value of said power being generated using the “Measure” feature, including power being generated from different MFC units to produce said data and/or said new data;
Display said data and/or said new data and/or said power being generated and/or said power being generated from different MFC units in at least one graphic format such that the user can compare power generation and/or growth behavior of at least one substrate-powered energy storage and generation system;
Compile said data and/or said new data and/or said power being generated and/or said power being generated from different MFC units in at least one file;
Communicate and/or transmit said at least one file in the form of at least one of e-mail, wire and/or wireless transmission, upload to at least one server and download from said at least one server to at least one user and/or at least one other person;
Display via visual presentation and/or audio presentation an educational illustrated comic that tells at least one story of an electric microbe comprising at least one storyline, said at least one storyline being retrievable via a user interaction with at least one button and/or touch screen location on at least one smart device, and said at least one storyline comprising at least one in-depth scientific explanation of at least one physical process, thus teaching at least one user about at least one scientific topic;
Communicate and/or transmit in the form of at least one of e-mail, wire and/or wireless transmission, upload to at least one server and download from said at least one server to at least one user and/or at least one other person at least one supplemental data and/or information comprising at least one of at least one link, common troubleshooting steps, tips and/or recommendations for increasing the MFC power generation.
